(19) United States
(12) Patent Application Publication
(10) Pub. No.: US 2008/0234576 A1
(43) Pub. Date: Sep. 25, 2008

(54) SYSTEM AND METHOD TO TRACK MOVEMENT OF A TOOL IN PERCUTANEOUS REPLACEMENT OF A HEART VALVE

(75) Inventors: Laurence Gavit-Houdant, New York, NY (US); Regis Vaillant, Villabon sur Yvette (FR); Elisabeth Soubelet, Meudon (FR)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/690,500

(22) Filed: Mar. 23, 2007

(51) Int. Cl. A61M 25/12

(52) U.S. Cl. 600/434; 623/2.11

(57) ABSTRACT

A system and method to track movement of a tool in percutaneous replacement of a heart valve of a subject is provided. The system includes an imaging system, a navigation system operable to track movement of the tool through the patient and to illustrate a representation of a position the tool in spatial relation relative to images acquired by the imaging system, and a controller. The controller is operable to identify one of a series of pathways to move the tool through a patient in percutaneous replacement of the heart valve, to identify a sequence of models illustrative of the one of the plurality of pathways, to detect the position of the tool within a threshold of one of the models in the sequence, and to generate display including a representation of the tool superimposed relative to the model within the threshold.

### BACKGROUND OF THE INVENTION

The subject matter described herein generally relates to tracking and medical imaging, and more particularly to a system and method to track movement of tool employed inpercutaneous replacement of a heart valve.

The heart valves are anatomical structures that prevent reflux of blood from a cavity of the heart to another, and that are therefore essential for good functioning of a heart. Known ways that a heart valve may dysfunction include stenosis of the heart valve and improper closure of the heart valve. Stenosis of the valve includes constriction of the heart valve such as to undesirably reduce blood flow. Improper closure of the blood valve can cause blood passing through to flow the wrong way. Typically, both above-described dysfunctions are corrected with replacement of the malfunctioning heart valve with a valve prosthesis.

A drawback of conventional approaches in deployment of a valve prosthesis includes difficulty in guiding the catheter device from its point of introduction into the patient to a point of deployment of the valve prosthesis in the heart. In another example, there is known difficulty in precisely position the valve-prosthesis using conventional imaging techniques so as avoid damage to surrounding anatomical structures of the patient. The certain conventional approach also includes positioning the valve prosthesis while the heart is temporarily placed in a frozen-like, generally immobilized state (e.g., temporarily rapidly pacing the heart). While the heart is placed in this generally immobilized state, it is undesired to inject markers or contrast agents that are commonly employed in conventional imaging techniques.

Thus, there is a need for a system and method to track movement of a tool through a patient in performing replacement of heart valve that addresses the drawbacks described above.

### BRIEF DESCRIPTION OF THE INVENTION

The above-mentioned needs are addressed by the embodiments described herein in the following description.

In accordance with one embodiment, a method to track movement of a tool in percutaneous replacement of a heart valve of a patient is provided. The method comprises the steps of identifying one of a series of pathways to move the tool through a patient in percutaneous replacement of the heart valve; identifying a sequence of models illustrative of the one of the series of pathways; tracking the movement of the tool through the patient; detecting a position of the tool within a threshold of the model; detecting the position of the tool within a threshold of one of the models in the sequence; and generating a display including a representation of the tool superimposed relative to the model within the threshold.

In accordance with another embodiment, a system operable to track movement of a tool in percutaneous replacement of a heart valve of a subject is provided. The system includes an imaging system operable to acquire a series of images; a navigation system operable to track movement of the tool through the patient and to illustrate a representation of a position of the tool in spatial relation to each of the series of images; and a controller in communication with the imaging system and the navigation system. The controller includes a processor in communication to execute a plurality of programmable instructions stored in a memory. The plurality of programmable instructions include identifying one of plurality of pathways to move the tool through a patient in percutaneous replacement of the heart valve, identifying a sequence of models illustrative of the one of the plurality of pathways, tracking the movement of the tool through the patient, detecting the position of the tool within a threshold of one of the models in the sequence, and generating a display including a representation of the tool superimposed relative to the model within the threshold.

In accordance with yet another embodiment, a computer program product that includes a series of computer-readable program instructions for execution by a processor to track movement of a tool through a subject in percutaneous replacement of a heart valve of a subject is provided. The plurality of computer-readable program instructions include identifying one of series of pathways to move the tool through a patient in percutaneous replacement of the heart valve; identifying a sequence of graphical representations illustrative of the one of the series of pathways; tracking the movement of the tool through the patient; detecting the position of the tool within a threshold of one of the models in the sequence, and displaying a representation of the tool superimposed relative to the model within the threshold.

Embodiments of varying scope are described herein. In addition to the aspects described in this summary, further aspects will become apparent by reference to the drawings and with reference to the detailed description that follows.

### BRIEF DESCRIPTION OF THE DRAWINGS

FIG. 1 is a schematic diagram illustrative of an embodiment of a system to track movement of a tool through a pathway of a patient in percutaneous replacement of a heart valve.

FIG. 2 is a flow diagram illustrative of an embodiment of a method to track movement of a tool through a pathway of a patient in percutaneous replacement of a heart valve.

FIG. 3 is a schematic representation of an embodiment of a model of at least a portion of a pathway to track movement of a tool in percutaneous replacement of a heart valve, the pathway, the pathway corresponding to an antegrade approach.

FIG. 4 is a schematic representation illustrative of another embodiment of a model of a pathway to track movement of a tool in percutaneous replacement of a heart valve, the pathway corresponding to a retrograde approach.

FIG. 5 is a schematic diagram of an embodiment of a method to identify a pathway to pass the tool through in percutaneous replacement of a heart valve.

FIG. 6 is a schematic diagram illustrative of an embodiment of a sequence of models correlated to the retrograde approach.

FIG. 7 is a schematic diagram illustrative of another embodiment of a sequence of models correlated to the retrograde approach.

FIG. 8 is a schematic diagram illustrative of an embodiment of a sequence of models correlated to the antegrade approach.

FIG. 9 is a schematic diagram illustrative of an embodiment of a method to generate a display of movement of tool in percutaneous replacement of the heart valve in the subject.

FIG. 10 is a schematic diagram illustrative of an embodiment of a sequence of models correlated to the antegrade approach in percutaneous repair of a mitral valve.

FIG. 11 is a schematic diagram illustrative of another embodiment of a sequence of models correlated to the antegrade approach in percutaneous repair of a mitral valve.

FIG. 12 is a schematic diagram illustrative of yet another embodiment of a sequence of models correlated to the antegrade approach in percutaneous repair of a mitral valve.

### DETAILED DESCRIPTION OF THE INVENTION

In the following detailed description, reference is made to the accompanying drawings that form a part hereof, and in which is shown by way of illustration specific embodiments, which may be practiced. These embodiments, although focused particularly on replacement of the aortic valve, are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken in a limiting sense.

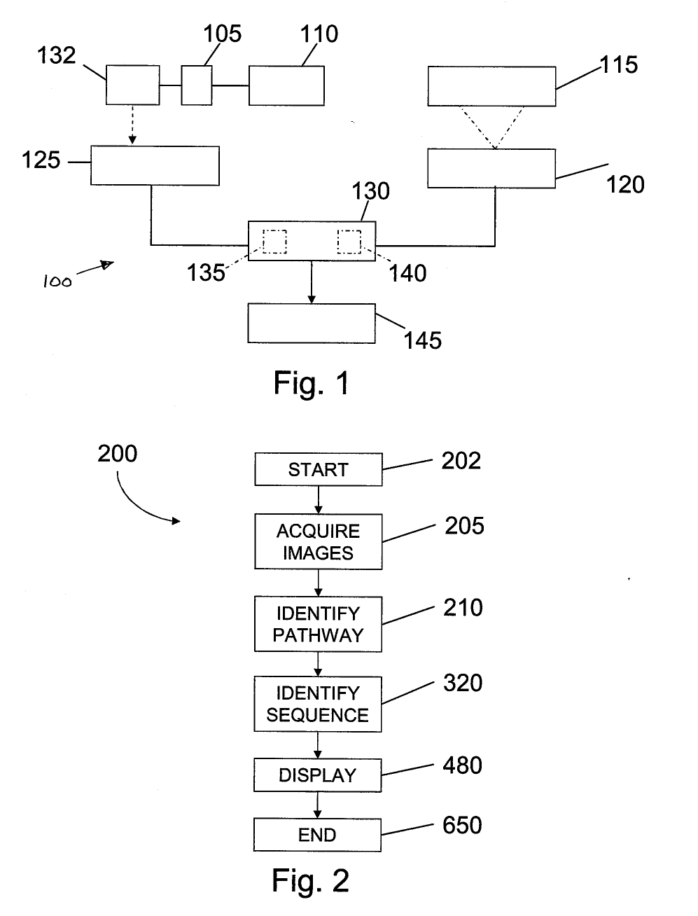

FIG. 1 illustrates an embodiment of a system 100 operable to track movement of a tool 105 in deployment of a valve prosthesis 110 in a subject 115. Examples of the tool 105 include a catheter or a guidewire, or other surgically invasive object. The following description specifically refers to the tool 105 operable to carry and deploy the valve prosthesis 110 in the heart of the subject 115. The system 100 includes an imaging system 120 and a navigation system 125 that in combination with a controller 130 creates an illustration of a representation of the tool 105 relative to the subject 115.

The imaging system 120 is generally operable to create a display of the pathway of the tool 105 traveling through the subject 115. Examples of the type of imaging system 120 include an electrocardiogram (ECG) tracking, magnetic resonance (MR) imaging, fluoroscopic imaging, computed tomography (CT) imaging, positron emission tomography (PET), x-ray imaging, ultrasound imaging, nuclear medicine enhanced imaging, etc. or combination of the above. The type of imaging system 120 can vary. The display is generally a two-dimensional, three-dimensional or four-dimensional model or re-constructed image of the pathway from the point of entry into the subject 115 to the location of deployment at the heart.

An example of an embodiment of the imaging system 120 is described in U.S. Patent Application No. 2006/0079759 to Vaillant et al., entitled “Method and apparatus for registering 3D models of anatomical regions of a heart and a tracking system with projection images of an interventional fluoroscopic system”, published on Apr. 13, 2006, and which is hereby incorporated herein by reference in its entirety. The imaging system 120 comprises a first image acquisition system configured to produce a fluoroscopy image of an anatomical portion of interest of the subject 115, and a second image acquisition system configured to produce a three-dimensional reconstructed image or model of the anatomical portion of the interest. An anatomical reference is defined to be common to both first and the second image acquisition systems, as well as the navigation system 125, such that the navigation system 125 is operable to register each of acquired images and the created three-dimensional images or models with reference in a conventional manner.

Still referring to FIG. 1, the navigation system 125 generally includes one or more sensors 132 operable to generate a signal (illustrated by dashed line) indicative of a location of the tool 105 relative to a reference. At least one sensor 132 is attached at the tool 105 or at the valve prosthesis 110 carried by the tool 105. Examples of the type of sensor 132 include radio frequency, electromagnetic, optical, etc., but the type of sensor can vary. The navigation system 125 is also operable to register the location of the sensor 132 relative to the images acquired by the imaging system 120.

The controller 130 generally includes a processor 135 generally operable to execute a series of programmable instructions stored in a memory 140. The type of memory 140 can include a hard-drive, a cd, a dvd, a memory stick or other type of product with a medium operable to store computer readable program instructions. The controller 130 is communication with both the imaging system 120 and the navigation system 125 and a display 145. Examples of the display include a monitor (e.g., LCD), a touch-screen, an audible speaker, LEDs, etc. The controller 130 can be an independent component, or integrated with one of the imaging system 120 and/or the navigation system 125.

Having provided the above-described system 100, the following is a general description of an embodiment of a method 200 (See FIG. 2) to track movement of the tool 105 employed in deployment of the valve prosthesis 110 in the subject 115. It should be understood that the foregoing sequence of acts or steps comprising the method 200 can vary in order, that the method 200 does not need to include each every act in the following description, and the method 200 can include additional acts not disclosed in the following description. One or more of the following acts comprising the method 200 can be represented as computer-readable programmable instructions for execution by a processor of the system 100 described above.

Referring to FIG. 2, step 202 is the start of the method 200. Step 205 includes acquiring a series of images via the imaging system 120 of a pathway through the subject 115 to pass the tool 105 and valve prosthesis 110. The number and type of images can vary. Step 210 includes identifying one of a series of approaches or pathways to move the tool 105 carrying the valve prosthesis 110 for deployment in the heart.

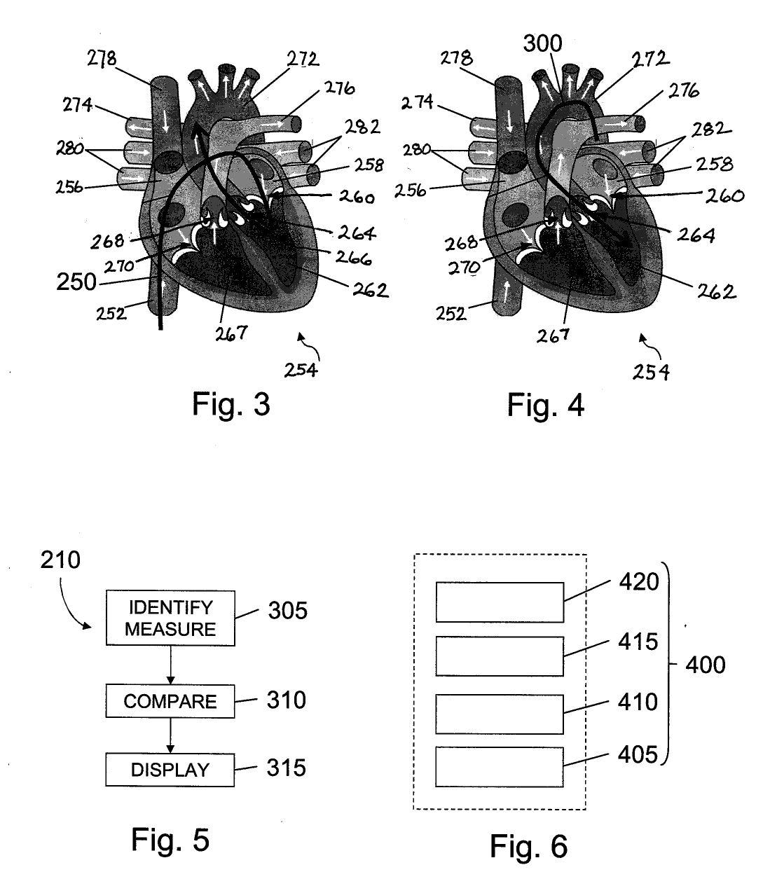

FIG. 3 generally illustrates a first embodiment of a pathway or approach, referred to as an antegrade approach 250, to pass the tool 105 through. The antegrade approach or pathway 250 includes a conduit of successive veins leading to the inferior vena cava 252 and chambers of a heart 254. The tool 105 to bring the valve prosthesis 110 to the heart may have a relatively large diameter. For example, the tool 105 can include a 24-Fr sheath having a diameter of about 8 mm. Therefore implantation and guiding of the tool 105 through the pathway 250 can be made easier by passing though generally larger sized veins, in comparison to arteries. However, a difficulty of this approach 250 includes obstacles encountered in navigating through the chambers of the heart 254. The embodiment of the antegrade approach 250 further includes creating a septal puncture between the right and left atria 256 and 258, respectively. Then, the tool 105 and valve prosthesis 110 is to be navigated via the mitral valve 260 into a left ventricle 262 and ultimately an aortic valve 264 of the heart 254 of the subject 115. This above-described U-turn manuever 266 increases opportunities to damage to the mitral cords that attach the mitral leaflets. Any damage to the mitral cords or mitral leaflets can result in an adverse cardiac event. Other anatomical structures illustrated in FIG. 3 include the right ventricle 267, the pulmonary valve 268, the tricuspid valve 270, the aorta 272, the right and left pulmonary arteries 274 and 276, the superior vena cava 278, and the right and left pulmonary veins 280 and 282 that may be used as anatomical landmarks in tracking the tool 105 through the heart 254.

FIG. 4 illustrates another embodiment of the pathway or approach, referred to as a retrograde approach 300. A difficulty of the retrograde approach 300 includes passing the tool 105 with the prosthesis 110 through a femoral artery (not shown), though an aortic arch (not shown), and crossing the aortic valve 264. Therefore, a threshold parameter in selecting the antegrade approach 300 includes a diameter (e.g., an inner diameter greater than 8 mm) of a femoral artery and iliac vessels (not shown) in the pathway 300 to the heart 254 in comparison to a diameter of the tool 105 and prosthesis 110. Another threshold parameter includes a level of calcification (or reduced diameter) along the pathway 300. Another difficulty of the antegrade approach 300 includes tracking movement of tool 105 and prosthesis 110 so as to manuever through an aortic arch so as to avoiding contact that creates a fragment of calcification that could travel to the brain.

Referring back to FIG. 2, an embodiment of step 210 includes characterizing or evaluating one or more of the pathways 250 and 300 in the percutaneous procedure and the defective valve to be replaced. Characterization includes identification of the pathway 250 and 300 in the acquired images, and measuring a level of calcification in a selected pathway 250 and 300 for the tool 105 and valve prosthesis 110 to travel through to the heart 254. Referring now to FIG. 5, an embodiment of step 210 includes step 305 of identifying and measuring a level of blockage or calcification along the pathway 250 and 300, and the step 310 of comparing the measured level blockage to a predetermined threshold (e.g., percent blockage or opening, diameter, etc.). Measurement of the level of blockage includes measurement of a morphology and dimensions of the veins and arteries comprising the pathway for the tool 105 to movement through to the heart 254. These measurements are compared to threshold parameters for travel of the tool 105 and valve prosthesis 110 therethrough. Step 315 includes providing a display illustrative of the measured values compared to the thresholds for the parameters for each of the pathways 250 and 300 to be viewed by the operator. The displaying step 315 can include illuminating or highlighting the pathway 250 or 300 that satisfies all of the thresholds, or alternatively the pathway 250 or 300 with measured parameters that exceed one or more thresholds. Referring back to FIG. 1, the series of images acquired with the imaging system 120 to track movement of the tool 105 along the pathway 250 and 300 can vary. In accordance with one example, the series of images includes a three-dimensional image or model reconstructed from a series of CT acquired images. An embodiment of a software that is operable to create the reconstructed three-dimensional image is INNOVA® 3D as manufactured by GENERAL ELECTRIC®. The software is also operable to measure a volume, a diameter, location and level of calcification, and general morphology of a vessel (e.g., vein, artery, etc.) or other anatomical structures comprising the pathway 250 and 300, as well as to compare the measured parameters relative to a threshold to pass the tool 105 through each of the series of pathways 250 and 300.

Referring now to FIG. 2, step 320 includes identifying an order or sequence of reconstructed three-dimensional images or models of the pathway 250 (FIG. 3) and 300 (FIG. 4) for illustration on the display. Each of the three-dimensional images or models represents a portion of a region of interest of an anatomical volume along the pathway 250 and 300. The anatomical volume can include one or more images of the heart 254 and/or surrounding anatomical organs or structures or combination thereof.

One embodiment of the series of three-dimensional images or models includes an illustration of all the anatomical structures through which the tool 105 passes, from the point of entry to the heart 254. However, the series of created three-dimensional images or models may be more or less so as to enhance monitoring or tracking of the tool 105 relative passing through certain anatomical structures with a morphology and/or dimensions identified with thresholds of increased difficulty in guiding the tool 105 therethrough, as well as to track movement of the tool 105 and deployment of the valve prosthesis 110 during predetermined steps (e.g., entry of tool 105 into the subject 115, positioning and deployment of the valve prosthesis 110, assessment of the deployment of the valve prosthesis 110, etc.) in percutaneous replacement of the defective valve of the heart 254.

Accordingly and as shown in FIG. 6, an embodiment of a sequence 400 of created three-dimensional images or models to track the retrograde approach or pathway 300 (FIG. 4) includes a first model 405 illustrative of the right atria of the heart 254, a second model 410 illustrative of the left atria of the heart 254, a third model 415 illustrative of the heart 254 valve to be replaced, and a fourth model 420 illustrative of the ventricle of the valve to be replaced.

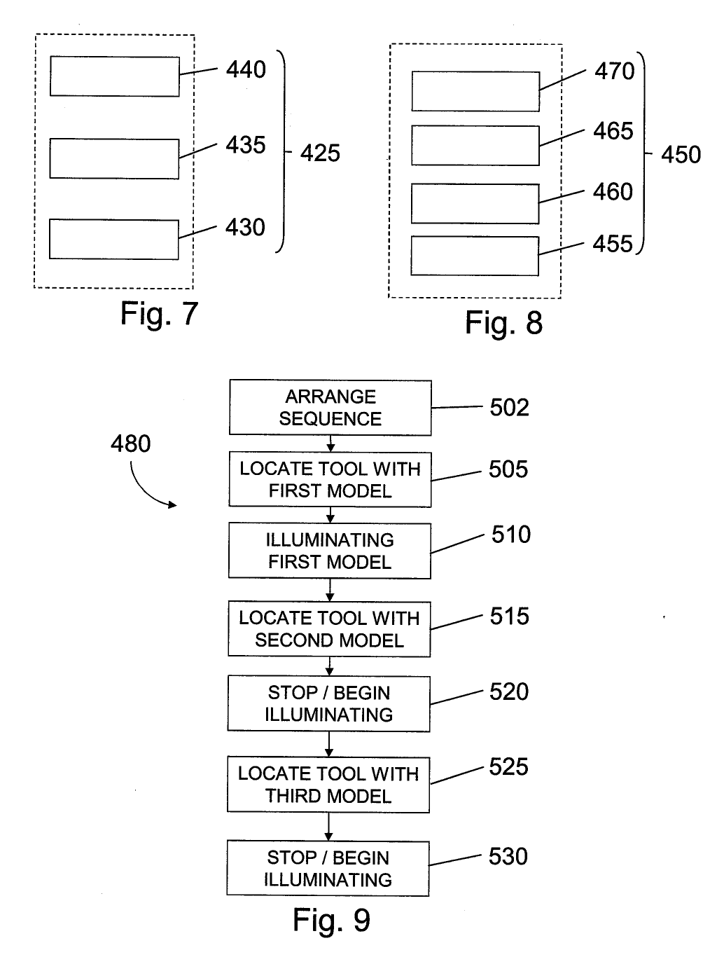

As shown in FIG. 7, another embodiment of a sequence 425 of created images or models so as to track the antegrade approach or pathway 250 includes a first model 430 is three-dimensional and illustrative of the iliac bifurcations. A second model 435 is three-dimensional and illustrative of the aortic arch. A third model 440 is two-or three-dimensional and illustrative of the valve to be replaced. The third model 440 is also illustrative of the ventricle where the valve being replaced is located.

As illustrated in FIG. 8, an embodiment of a sequence 450 of created images or models to track the antegrade approach or pathway 250 (FIG. 3) includes a first model 455 illustrative of arterial iliac bifurcations of the subject 115, a second model 460 illustrative of the aortic arch, a third model 465 illustrative of the heart valve to be replaced, and a fourth model 470 illustrative of the corresponding ventricle of the heart valve to be replaced. All of the models 455, 460, 465 and 470 are three-dimensional.

Although particular embodiments of the sequences 400, 425 and 450 are described above, it should be understood that alternative sequences can include various types (e.g., two-dimensional, three-dimensional, four-dimensional, etc. or combinations thereof).

Referring back to FIG. 2, step 480 includes displaying the sequence of images or models corresponding to the selected pathway 250 and 300 (See FIGS. 3 and 4, respectively) to pass the tool 105 and valve prosthesis 110 through the subject 115 (FIG. 1). As the physician passes the tool 105 and prosthesis 110 through the selected pathway 250 and 300 in percutaneous replacement of the valve in the heart 254 of the subject 115, the system 100 creates a display illustrative of a representation of the tool in spatial relation to each of the sequence of images or models. In one example, the imaging system 120 includes a CT imaging system operable to combine or fuse or superimpose each three-dimensional model or image of the sequence with an image of the corresponding anatomical region from a fluoroscopy system, so that a representation of the tool 105 may be superimposed with each model or image of the sequence. The navigation system 125 tracks a position of the tool 105 and valve prosthesis 110. The navigation system 125 enables a practitioner to precisely follow movement of the tool 105 relative to the illustration of anatomical structures in the sequence of reconstructed images or models, the anatomical structures corresponding to the critical locations of the pathway 250 and 300 corresponding to the selected for replacement of the valve.

In accordance with one embodiment, the step of 480 of displaying any of the sequence 400, 425 and 450 of images is correlated simultaneously with a position of the tool 105 as moves in steps 240 and 245, where displaying of the three-dimensional models depends on the location of the tool 105 as tracked by the navigation system 125 as the tool 105 moves through the subject 115. Therefore, the step 480 of displaying any of the sequence 400, 425 and 450 of images is complementary to moving and tracking the tool 105. For example, in accordance with the sequence 450 of images correlated to the antegrade approach 250, the step 480 includes increased illumination of model 455 (including an illustration of the tool 105 relative thereto) relative to the other models 460, 465, and 470 of the sequence 450 that are outside a threshold distance of the tool 105, as tracked by the navigation system 125. Accordingly, a technical effect is that all of the models are simultaneously illustrated for viewing on the display, but illumination of each of the models 460, 465, and 470 is increased relative to the others by a predetermined threshold as a representation of the tool 105 is displayed simultaneously therewith and relative thereto. The difference in illumination of the models 460, 465, and 470 can be changed by at the controller 130 or the imaging system 120 or navigation system 125. The increased illumination of each of the models 455, 460, 465, and 470 relative to one another can be in response to detecting movement of the tool 105 within a predetermined threshold distance of the respective model 455, 460, 465, and 470 or anatomical landmarks in the model 455, 460, 465, and 470. Of course, a similar approach can be used with illumination of the other sequences 400 and 425 described above.

FIG. 9 includes a schematic diagram illustrative of another example of the step of displaying 480 is performed simultaneously and correlated with the tracking of the tool 105 relative thereto with reference to the antegrade approach 250 described above. Although the displaying step 480 is described with reference to the antegrade approach 250, it should be understood that the following description can also be applicable to the retrograde approach 300 or other approaches not described herein. Step 502 includes generating a display that includes a spatial arrangement of the models 455, 460, 465, and 470 in the sequence 450 relative to one another in accordance to a direction of the selected pathway or approach 250 through the subject 115. Step 505 includes detecting a location of the tool 105 in the subject 115 and within a view or threshold distance of the first model illustrative of the iliac bifurcations. In response to the detecting step 505, step 510 includes simultaneously displaying the location of the tool 105 and/or prosthesis 110 superimposed in spatial relation with the first model 455 illustrative of the iliac bifurcations.

Still referring to FIG. 9, step 515 includes detecting a location of the tool 105 and/or prosthesis 110 within a view or threshold distance of the second model 460 illustrative of the right and left atria 256 and 258 of the heart 254. In response to the detecting step 515, step 520 includes stopping display of the first model 455 and beginning display of a representation of the tool 105 superimposed with the second model 460. The superimposed display of the tool 105 and second model 460 is to guide a physician in performing the septal puncture from the atria to the left ventricle 262 of the heart 254.

Step 525 includes detecting a location of the tool 105 within a view or threshold distance of the third model 465 representative of the left ventricle 262 of the heart 254. In response to the detecting step 525, step 530 includes stopping display of the tool 105 with the second model 460 and beginning display of the representation of the tool 105 superimposed in spatial relation with the third model 465. The superimposed display of the tool 105 with the third model 465 is a guide for a physician to move the tool 105 in replacement of the diseased valve with the prosthesis 110 relative to surrounding calcifications and during a short time slot of rapid pacing of the patient's heart 254. Of course, the above-described steps 505, 510, 515, 520, 525, and 530 are similar in tracking displaying a representation of the tool 105 relative to the other model 470 in the sequence 450. Referring back to FIG. 2, step 650 is the end of the method 200. Also, it should be understood that the sequences 400, 425, and 450 can include additional models.

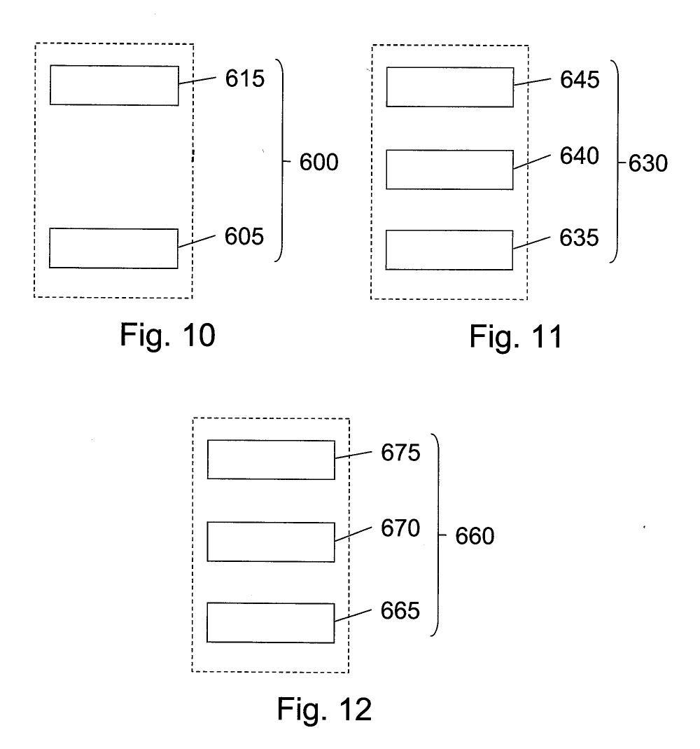

Although the above detailed description is in reference to percutaneous replacement of the aortic valve, It should be understood that the subject matter applicable to replacement or repair of other valves (e.g., mitral valve 260, the tricuspid valve, the pulmonary valve, etc.). For example and as illustrated in FIG. 10, repair of the mitral valve 260 can include identifying a sequence 600 of images or models for the antegrade approach 300. An embodiment of the sequence 600 includes a first model 605 illustrative of the right and left atria 256 and 258, and a second model 615 illustrative of both the mitral valve 260 and a left ventricle 262 of the heart 254.

Alternatively and as shown in FIG. 11, an embodiment of a sequence 630 of images is correlated to a Coronary Sinus approach. This sequence 630 of images includes a first model 635 illustrative of the coronary sinus (not shown), a second model 640 illustrative of the left and right atria 256 and 258, and a third model 645 illustrative of coronary circumflex artery (not shown). FIG. 12 illustrates yet another embodiment of a sequence 660 of images that is correlated to a trans-ventricular approach. This sequence 660 of images includes a first model 665 illustrative of the arterial iliac bifurcation to guide introduction of the tool 105 into the subject 115, a second model 670 illustrative of the aortic arch (not shown), and a third model 675 illustrative of the left ventricle 262 and left atrium 258 to guide movement and anchorage of the prosthesis valve carried by the tool 105.

Although the method 200 is described with reference to the antegrade approach 250 and sequence 450, it should be understood that the method 200 is applicable to each of the other approaches and sequences described above or combinations thereof or with other approaches or pathways not described herein.

This written description uses examples to disclose the subject matter, including the best mode, and also to enable any person skilled in the art to make and use the subject matter described herein. The scope of the subject matter described herein is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

We claim:

1. A method to track movement of a tool in percutaneous replacement of a valve of a heart of a subject, the method comprising the steps of:
identifying one of plurality of pathways to move the tool through a patient in percutaneous replacement of the heart valve;
identifying a sequence of models correlated to and illustrative of the one of the plurality of pathways;
tracking the movement of the tool through the patient;
detecting the position of the tool within a threshold of one of the models in the sequence; and
generating a display including a representation of the tool superimposed in spatial relation to the model within the threshold.

2. The method of claim 1, wherein one of the plurality of pathways is a retrograde approach, and wherein the sequence of images correlated to the retrograde approach includes a first model illustrative of arterial iliac bifurcations of the subject, a second model illustrative of the aortic arch, a third model illustrative of the heart valve to be replaced, and a fourth model illustrative of the corresponding ventricle of the heart valve to be replaced, and wherein all of the models are three-dimensional.

3. The method of claim 1, wherein one of the plurality of pathways is an antegrade approach, and wherein the sequence of images correlated to the antegrade approach includes a first model illustrative of the right atria of the heart, a second model illustrative of the left atria of the heart, a third model illustrative of the heart valve to be replaced, and a fourth model illustrative of the ventricle of the valve to be replaced.

4. The method of claim 1, wherein the generating a display step further includes:
detecting the position of the tool within a threshold of a first model in the sequence;
illustrating the representation of the tool in spatial relation relative to the first model;
detecting the position of the tool within a threshold of a second model in the sequence; and
automatically removing the first model from the display and automatically illustrating the representation of the tool in spatial relation relative to the second model.

5. The method of claim 4, the generating the display step further comprising the steps of:
detecting the position of the tool within a threshold of a third model in the sequence; and
automatically removing the second model from the display and automatically illustrating the representation of the tool in spatial relation relative to the third model.

6. The method of claim 1, wherein each of the models in the sequence are spatially arranged on a display relative to one another in accordance to a direction of the selected pathway.

7. The method of claim 1, wherein the sequence comprises a first model illustrative of a left atrium of the heart, a second model illustrative of the coronary sinus, and a third model illustrative of a circumflex coronary artery, and a fourth model illustrative of the mitral valve to be replaced.

8. The method of claim 1, wherein the sequence comprises a first model illustrative of the left or right illiac bifurcation, a second model illustrative of a aorta, a third model illustrative of both an aortic valve and a left ventricle, a fourth model illustrative of a mitral annulus, a mitral valve and a left atrium of the subject.

9. The method of claim 1, wherein the identifying step includes:
measuring a level of blockage along at least one of the plurality of pathways;
comparing a level blockage to a predetermined threshold of blockage; and
selecting another of the plurality of pathways to pass the tool if the level of blockage exceeds the predetermined threshold of blockage.

10. A system operable to track movement of a tool in percutaneous replacement of a heart valve of a subject, comprising:
an imaging system operable to acquire a plurality of images;
a navigation system operable to track movement of the tool through the patient and to illustrate a representation of a position the tool in spatial relation to each of the plurality of images; and
a controller in communication with the imaging system and the navigation system, the controller including a processor in communication to execute a plurality of programmable instructions stored in a memory, the plurality of programmable instructions including:
identifying one of plurality of pathways to move the tool through a patient in percutaneous replacement of the heart valve,
identifying a sequence of models illustrative of the one of the plurality of pathways,
tracking the movement of the tool through the patient,
detecting the position of the tool within a threshold of one of the models in the sequence, and
generating a display including a representation of the tool superimposed relative to the model within the threshold.

11. The system of claim 10, wherein one of the plurality of pathways is a retrograde approach, and wherein the sequence of images correlated to the retrograde approach includes a first model illustrative of arterial iliac bifurcations of the subject, a second model illustrative of the aortic arch, a third model illustrative of the heart valve to be replaced, and a fourth model illustrative of the corresponding ventricle of the heart valve to be replaced, and wherein all of the models are three-dimensional.

12. The system of claim 10, wherein one of the plurality of pathways is an antegrade approach, and wherein the sequence of images correlated to the antegrade approach includes a first model illustrative of the right atria of the heart, a second model illustrative of the left atria of the heart, a third model illustrative of the heart valve to be replaced, and a fourth model illustrative of the ventricle of the valve to be replaced.

13. The system of claim 10, wherein the step of generating the display includes:
detecting the position of the tool within a view of a first model in the sequence;
illustrating a representation of the tool superimposed in spatial relation relative to the first model;
detecting the position of the tool within a field of view of a second model in the sequence; and
automatically removing the first model from the display and automatically illustrating the representation of the tool in spatial relation relative to the second model.

14. The system of claim 13, the generating the display step further comprising the steps of:
detecting the position of the tool within a field of view of a third model in the sequence; and
automatically removing the second model from the display and automatically illustrating the representation of the tool in spatial relation relative to the third model.

15. The system of claim 14, wherein the first, second and third models are arranged in accordance to a direction along the one of the selected pathways relative to one another.

16. The system of claim 10, wherein the sequence comprises a first model illustrative of a left atrium of the heart, a second model illustrative of the coronary sinus, and a third model illustrative of a circumflex coronary artery, and a fourth model illustrative of the mitral valve to be replaced.

17. The system of claim 10, wherein the sequence comprises a first model illustrative of the left or right illiac bifurcation, a second model illustrative of a aorta, a third model illustrative of both an aortic valve and a left ventricle, a fourth model illustrative of a mitral annulus, a mitral valve and a left atrium of the subject.

18. The system of claim 10, wherein the identifying step includes:
measuring a level of blockage along at least one of the plurality of pathways;
comparing a level blockage to a predetermined threshold to pass the tool through; and
selecting another of the plurality of pathways to pass the tool if the level of blockage exceeds the predetermined threshold.

19. A computer program product comprising a plurality of computer-readable program instructions for execution by a processor to track movement of a tool through a subject in percutaneous replacement of a heart valve of a subject, the plurality of computer-readable program instructions including:
identifying one of plurality of pathways to move the tool through a patient in percutaneous replacement of the heart valve;
identifying a sequence of graphical representations illustrative of the one of the plurality of pathways;
detecting the position of the tool within a threshold of one of the models in the sequence, and
generating a display including a representation of the tool superimposed relative to the model within the threshold.

20. The computer program product of claim 19, wherein the illustrating step includes:
detecting the position of the tool within a view of a first model in the sequence;
illustrating a representation of the tool superimposed in spatial relation relative to the first model;
detecting the position of the tool within a field of view of a second model in the sequence; and
automatically removing the first model from the display and automatically illustrating the representation of the tool in spatial relation relative to the second model.
